(19) United States
(12) Patent Application Publication
(10) Pub. No.: US 2005/0118619 A1
(43) Pub. Date: Jun. 2, 2005

(54) Dark quenchers for fluorescence resonance energy transfer (FRET) in bioassays

(76) Inventors: Wensheng Xia, Santa Fe, NM (US); David Whitten, Albuquerque, NM (US); Duncan McBranch, Santa Fe, NM (US)

Correspondence Address: PIPER RUDNICK LLP, 1200 Nineteenth Street, N.W., Washington, DC, 20036-2412, US

(21) Appl. No.: 10/945,097

(22) Filed: Sep. 21, 2004

Related U.S. Application Data
(60) Provisional application No. 60/504,437, filed on Sep. 22, 2003.

(51) Int. Cl. C12Q 1/68; G01N 33/53; C07D 43/02

(52) U.S. Cl. 435/6; 534/727; 534/770; 435/7.1

(57) ABSTRACT

Non-fluorescent dyes (i.e., dark quenchers) which can be used to quench the fluorescence of energy donors in bioassays through fluorescence resonance energy transfer (FRET) are described. The dark quenchers can be associated with (e.g., conjugated to) peptides, proteins, antibodies, DNA/RNA, or other biological molecules or receptors or complexed to metal containing compounds to develop bioassays based on donor-acceptor energy transfer. Bioassays are also described wherein an increase or a decrease in separation distance between a fluorescent donor compound and a dark quencher or dark quencher conjugate is detected. Kits including the dark quenchers or dark quencher conjugates are also described.

This application claims the benefit of U.S. Provisional Patent Application Ser. No. 60/504,437, filed Sep. 22, 2003, which application is incorporated by reference herein in its entirety.

### BACKGROUND

1. Technical Field

The present application relates generally to bioassays and reagents for use in bioassays. In particular, the present application relates to dark quenchers which can be used to quench the fluorescence of energy donors in bioassays through fluorescence resonance energy transfer (FRET) and to bioassays employing the dark quenchers.

2. Background of the Technology

Rapid advances in molecular biology have led to the identification of increasing numbers of substances (e.g., enzymes, cytokines, and nucleic acids) which play key roles in the function of both normal and stressed systems. Many techniques have been used to detect biological analytes including radioactive labeling, various immunoassays including ELISA (enzyme-linked immunosorbent assays) chemiluminescence and various fluorescence-based techniques. Of particular interest, fluorescence resonance energy transfer (FRET) has been extensively used to assay many biological analytes (proteins, antibodies, DNA/RNA etc.) in applications ranging from detection to high throughput screening (HTS) for dug discovery.

Many organic dyes may be used as quenchers in FRET bioassays as long as the spectrally matched fluorophore-quencher pairs can be brought to close proximity with proper alignment. However, many organic dyes which might be used as quenchers have intrinsic fluorescence, which can result in high background fluorescence (through energy transfer) and hence attenuate the sensitivity of FRET assays. Dark quenchers with little or no intrinsic fluorescence can efficiently quench the fluorescence from the proximate fluorophores with little background. Of many dark quenchers, 4-(4′dimethylaminophenylazo)benzoic acid (DABCYL) is a common dark quencher used widely in many assays, such as “molecular beacons” for DNA detection (U.S. Pat. No. 5,989,823). However, the limited absorption range for DABCYL quenchers restricts the utility of these compounds by allowing the use of a limited number of fluorophores as donors. Diazo dyes of the BHQ series, which are referred to as “Black Hole Quenchers” (International Patent Publication No. WO 01/86001), provide a broad range of absorption which overlaps well with the emission of many fluorophores. The QSY series dyes from Molecular Probes are another series of dark quenchers used extensively as quenching reagents in many bioassays (U.S. Pat. No. 6,399,392). All three of these dark quencher families have a common limitation: high hydrophobicity and poor water-solubility. The poor water solubility limits their uses in many ways, both by decreasing the solubility of the dye-conjugated biomolecules used in the assays and by making the preparation and purification very difficult. Additionally, the high hydrophobicity of these dyes may result in a high level of non-specific association with biomolecules in many protein, peptide and DNA assays. One class of relatively water-soluble dyes is the non-fluorescent asymmetric cyanine dye series (See, for example, U.S. Pat. No. 6,348,596).

Accordingly, there still exists a for improved quenchers for FRET bioassays having higher water solubility which can be used in rapid and highly specific methods for detecting and quantifying chemical, biochemical and biological substances.

### SUMMARY

According to a first embodiment of the invention, a compound is provided having a general structure as set forth in formulae (Ia), (1b) or (II) below: 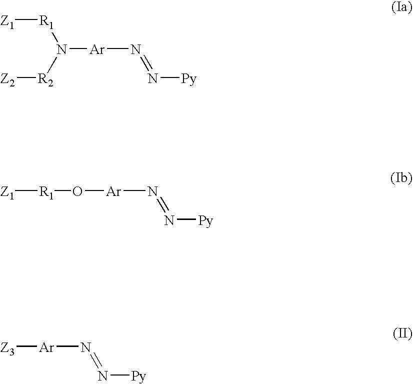
wherein:

Ar is a substituted or non-substituted aryl group;

Py is a substituted or non-substituted hetero-aromatic ring;

R₁ and R₂ independently represent a C₁ to C₄ alkyl chain or hydrogen;

Z₁ and Z₂ independently represent a substituted or non-substituted sulfonate, phosphate or carboxylate, pentafluorophenyl ester, p-nitrophenylester, or a moiety represented by one of the following formulae: 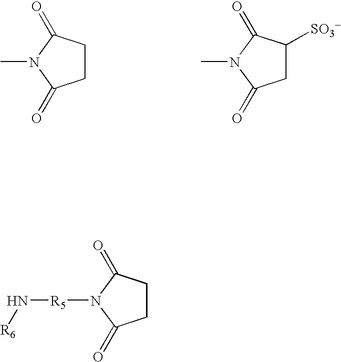
wherein R₅ and R₆ are alkyl groups; and

Z₃ is OH, OR₇, NH₂, NHAr′ or NAr′₂, SH, SR₇, or SCN wherein Z₃ is at the ortho-position of the aryl group Ar, Ar′ is an aromatic or hetroaromatic ring and R₇ is an alkyl or aromatic group.

Exemplary compounds include compounds having a general structure as set forth in formulae (IIIa), (IIIb) or (IV) below: 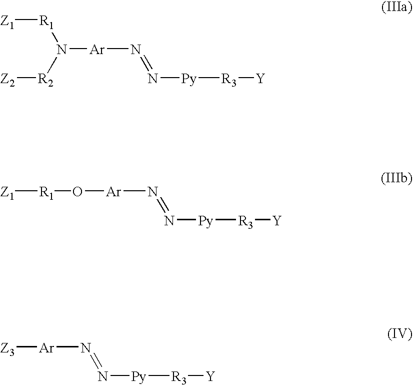
wherein:

R₃ is a C₁ to C₈ alkyl chain; and

Y is: —COOH, —SH, —OH, isocyanate, epoxide, iodoacetate, bromoacetate, NR′R″ where R′ and R″ are hydrogen or alkyl or aromatic rings, or —COOR₄ wherein R₄ is pentafluorophenyl ester, p-nitrophenylester, or a moiety represented by one of the following formulae: 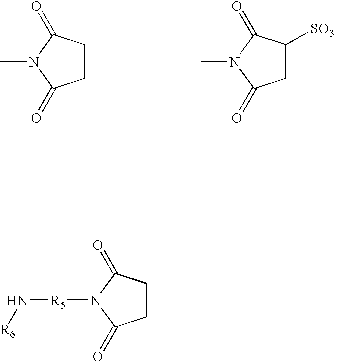
wherein R₅ and R₆ are alkyl groups or wherein Y is a moiety represented by the following formula:
—OP(OR₈)(N(R₉)₂)₂
wherein, R₈ and R₉ are independently alkyl or substituted alkyl groups. According to a preferred embodiment, R₈ is cyanoethyl and R₉ is isopropyl.

Exemplary specific compounds of the above type include compounds represented by either of the following formulae:

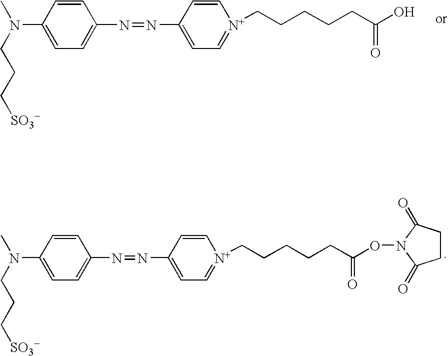

Conjugates of a quencher compound having a structure as set forth above and a biomolecule are also provided. The biomolecule conjugated to the quencher compound can be a polypeptide, a protein, an antibody, or a nucleic acid (e.g., DNA or RNA).

According to further embodiments, a bioassay is provided in which an increase or a decrease in separation distance between a donor fluorescent moiety and a dark quencher or dark quencher conjugate as set forth above is detected.

According to another embodiment, a kit comprising a dark quencher or a dark quencher conjugate as set forth above is also provided.

### BRIEF DESCRIPTION OF THE DRAWINGS

FIG. 1 shows a synthetic route for the preparation of a dark quencher as described in the present application.

FIG. 2 is a graph showing the absorption spectrum of the compound shown in FIG. 1 in aqueous PBS (phosphate buffer saline) solution.

FIG. 3 illustrates a reaction scheme for forming dark quencher-metal complexes.

### DETAILED DESCRIPTION

The present application relates to non-fluorescent dyes (i.e., dark quenchers which can be conjugated to or associated biological molecules (e.g., peptides, proteins, antibodies, DNA/RNA) or other receptors to develop bioassays based on donor-acceptor energy transfer. These non-fluorescent dyes are highly water soluble and functionalized to allow their rapid attachment to many biological targets. The high molar extinction coefficients and broad absorption spectra of these dark quenchers make them ideal for quenching donor fluorescence without generating background emission.

Moreover, the present invention provides a class of dark quenchers with excellent water solubility and a broad range of absorption spectra covering the emission spanning most fluorescent dye donors ranging from individual fluorescent dyes to fluorescent polymers or fluorescent polymer ensembles. These dark quenchers are easy to prepare and can be functionalized to afford conjugates with many biological macromolecules including peptides, proteins, antibodies, and nucleic acids (e.g., DNA or RNA).

Descriptions of Exemplary Dark Quenchers

Exemplary dark quenchers described herein are a series of azopyridinium dyes able to quench many fluorophores efficiently with little to no background, including fluorescein, rhodamine, Texas Red, Quantum Dots, cyanine dyes and their derivatives, Alexa Fluor dyes, BODIPY dyes, fluorescent polymers and polymer ensembles and fluorescent proteins such as phycoerythrin. These dark quenchers typically exhibit absorption from 450˜700 nm with high solubility in aqueous media. These dyes can also be functionalized with a variety of reactive groups which can afford selective reaction with many biological species through different coupling chemistry.

The dark quenchers described herein are zwitterionic azopyridinium compounds. These compounds have a general structure as set forth in formulae (Ia), (1b) or (II) below: 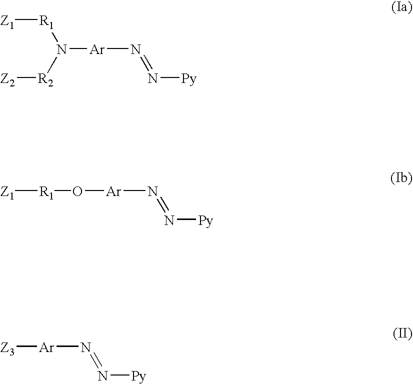
wherein:

Ar is a substituted or non-substituted aryl group;

Py is a substituted or non-substituted hetero-aromatic ring;

R₁ and R₂ independently represent a C₁ to C₄ alkyl chain or hydrogen;

Z₁ and Z₂ independently represent a substituted or non-substituted sulfonate, phosphate or carboxylate, pentafluorophenyl ester, p-nitrophenylester, or a moiety represented by one of the following formulae: 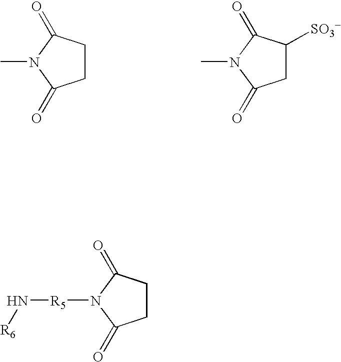
wherein R₅ and R₆ are alkyl groups; and

Z₃ is OH, OR₇, NH₂, NHAr′ or NAr′₂, SH, SR₇ or SCN wherein Z₃ is at the ortho-position of the aryl group Ar, Ar′ is an aromatic or hetroaromatic ring and R₇ is an alkyl or aromatic group.

Exemplary compounds include compounds having a general structure as set forth in formulae (IIIa), (IIIb) or (IV) below: 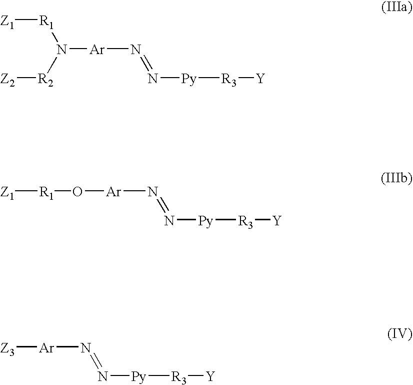
wherein:

R₃ is a C₁ to C₈ alkyl chain; and

Y is: —COOH, —SH, —OH, isocyanate, epoxide, iodoacetate, bromoacetate, NR′R″ where R′ and R″ are hydrogen or alkyl or aromatic rings, or —COOR₄ wherein R₄ is pentafluorophenyl ester, p-nitrophenylester, or a moiety represented by one of the following formulae: 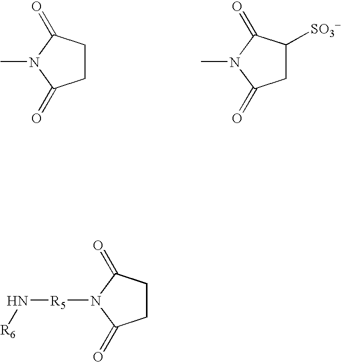
wherein R₅ and R₆ are alkyl groups or wherein Y is a moiety represented by the following formula:
—OP(OR₈)(N(R₈)₂)₂
wherein, R₈ and R₉ are independently alkyl or substituted alkyl groups. According to a preferred embodiment, R₈ is cyanoethyl and R₉ is isopropyl.

Specific exemplary compounds include the compounds represented by either of the following formulae: 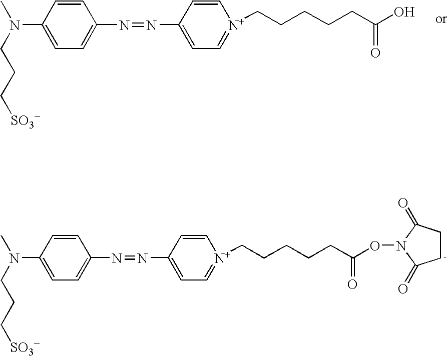
Quencher-Biomolecule Bioconjugates

Dark quenchers as described above can be conjugated to (e.g., reacted with) a biological molecule (i.e., a biological target) to form a bioconjugate. Exemplary biological targets include, but are not limited to:

1. Polypeptides: either the N-terminal or the C-terminal of a polypeptide can be reacted with the dark quenchers though EDC (i.e., 1-[3-(Dimethylamino)-propyl]-3-ethylcarbodiimide hydrochloride) or HOBT (1-Hydroxybenzotriazole) activation reaction of carboxylate or NHS (N-Hydroxysuccinimide) reaction with amino groups. Alternatively, a cysteine containing peptide can be directly reacted with a maleimide or α-halo carbonyl containing dark quenching compound to form a bioconjugate. The polypeptide can contain an enzyme cleavable sequence or a substrate with a certain sequence which is capable of being phosphorylated or dephosphorylated through the reaction mediated by specific enzymes. The polypeptide can also be a target for an antibody.

2. Antibodies: the dark quenchers can be conjugated with various antibodies though amide chemistry, isocyanate chemistry, thiol chemistry, epoxide chemistry etc. The antibody could be either a whole antibody or a cleaved (F_{ab} or F_{c}) antibody fragment.

3. Proteins: the dark quenchers can be conjugated with various proteins though, for example, amide chemistry, isocyanate chemistry, thiol chemistry, or epoxide chemistry. Proteins containing no thiol groups can be conjugated through hetero-linkage reagents.

4. Nucleic acids: the dark quenchers can be conjugated to various nucleic acids including DNA or RNA sequences though, for example, amide chemistry, isocyanate chemistry, thiol chemistry or phosphine chemistry;

5. Biotin: the dark quenchers can be conjugated with various biotin or biotin-PEG (polyethylene glycol) reagents though, for example, amide chemistry, isocyanate chemistry or thiol chemistry.

6. Biotin-avidin complex: biotin-dark quencher conjugates together with other biotinylated proteins can form co-complexes with avidin analogues (e.g., avidin, streptavidin or neutravidin) to make dye-protein complexes.

Dark Quencher Synthesis

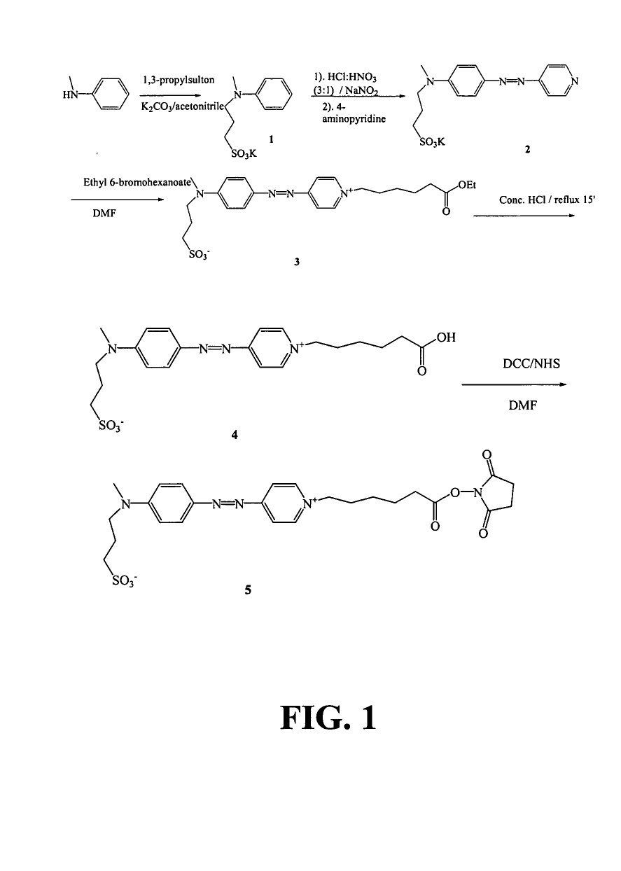

A synthesis route for a dark quencher according to one embodiment is shown in FIG. 1. The synthesis of both an azo-COOH (4) and an azo-NHS (5) form of the dark quencher is shown in FIG. 1. Both the azo-COOH (4) and the azo-NHS (5) forms of the dark quencher can be reacted with biomolecules having amino groups.

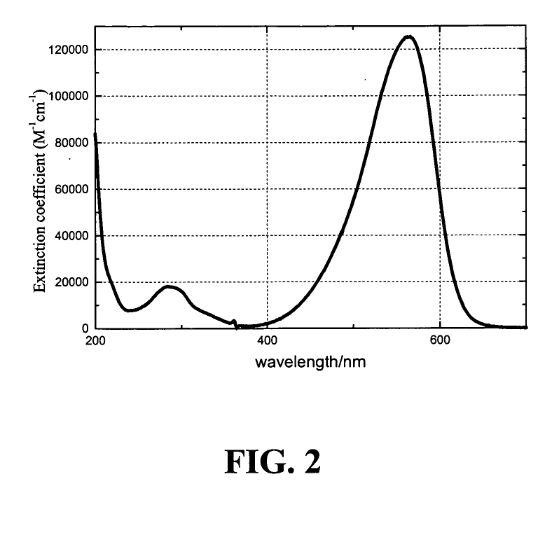

The absorption spectrum in PBS of the dark quencher synthesized in FIG. 1 is shown in FIG. 2. As can be seen from FIG. 2, the molar extinction coefficient is about 125,000 cm⁻¹ and the dark quencher has a maximum absorption of about 560 nm.

Quencher-Metal Complexes

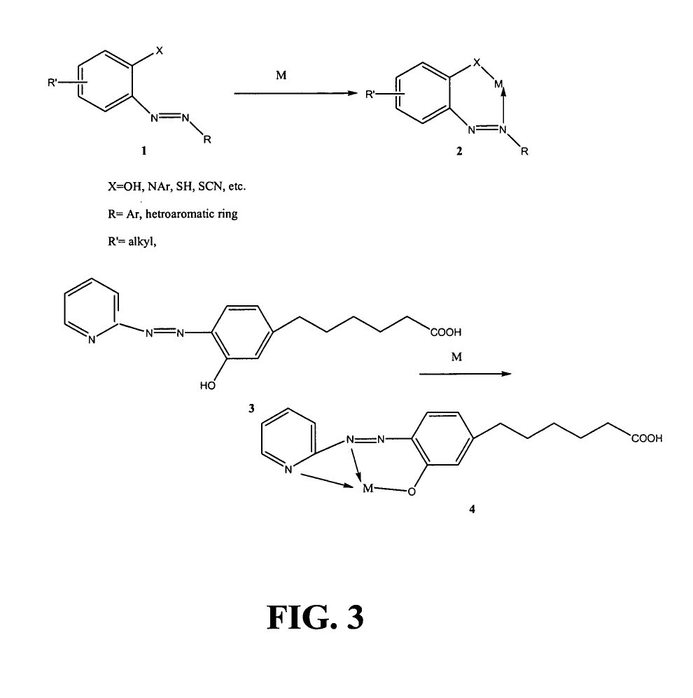

The azo-based dark quenchers also may be used to form complexes with metal containing compounds (e.g., gallium containing compounds). An exemplary complex of this type is shown in FIG. 3. As shown in FIG. 3, metal complexes 2 and 4 are formed from dark quenchers 1 and 3. In FIG. 3, “M” represents a trivalent or tetravalent metal ion or metal complex. Metal complexes 2 and 4 retain a ligand binding site that, depending on the metal, may associate specifically with ligands, often with very high binding constants. This can provide the basis for biosensing applications using fluorophores (e.g., fluorescent polymers, fluorescent proteins, quantum dots, etc.) conjugated with a peptide, protein, enzyme, or DNA/RNA component and containing a ligand for the metal in structures 2 and 4. Moreover, dye-metal complexes 2 and 4 may be used as a specific interaction probe in bio-recognition or bioassays. When metal-ligand association occurs, the fluorophore will be quenched. These conjugates may be used both in assays of reactions in which the ligand is either produced or consumed as well as in competition assays.

Applications

The dark quenchers or conjugates of the dark quenchers described herein can be used in bioassays. In particular, increases or decreases in separation distance between a fluorescent donor and a dark quenching compound acceptor can be detected using a dark quencher or bioconjugates comprising a dark quencher as described herein.

Any assay that relies upon the measurement of the proximity of fluorescent donors and quenching compounds in a system may be carried out using dark quenchers as described herein. Assays of this type can be used to detect and/or quantify an increase or a decrease in the separation distance of a luminophore donor and a dark quenching compound acceptor.

In one embodiment, an assay can be used to detect molecular or structural assembly. In another embodiment, an assay can be used to detect molecular or structural disassembly. In yet another embodiment, an assay can be used to detect a conformational change in a molecule, macromolecule or structure.

The luminescence of a fluorescent donor can be quenched upon being placed in close proximity to a dark quenching compound as described herein. Exemplary systems which can be analyzed include: protein subunit assembly; enzyme-mediated protein assembly; molecular dimensions of proteins; membrane-protein interactions; protein-protein interactions; protein-protein-nucleic acid complex assembly; receptor/ligand interactions; immunoassays; nucleic acid hybridizations; quantitative detection of specific DNA sequence amplification; detection of DNA duplex winding; nucleic acid-protein interactions; nucleic acid-drug interactions; primer extension assays for mutation detection; reverse transcriptase assay; strand exchange in DNA recombination reactions; membrane fusion assays; transmembrane potential sensing; and ligation assays.

In particular, specific binding pair members labeled with a dark quenching compound can be used as probes for the complementary member of that specific binding pair. The complementary member is typically labeled with a fluorescent label and association of the two members of the specific binding pair results in quenching of luminescence. This assay is particularly useful in nucleic acid hybridization assays, evaluation of protein-nucleic acid interaction, and in immunoassays.

In one embodiment, a loss of luminescence indicates the association of an enzyme with an enzyme substrate, agonist or antagonist, such that the luminophore on one member of the interacting pair is brought into close proximity to a dark quenching compound on the other. Exemplary specific binding pair members include proteins that bind non-covalently to low molecular weight ligands (including biotin), oligonucleotides, and drug-haptens. Representative specific binding pairs include: antigen/antibody; biotin/avidin, streptavidin, anti-biotin; folate/folate-binding protein; IgG/protein A or protein G; drug/drug receptor; toxin/toxin receptor; carbohydrate/lectin or carbohydrate receptor; peptide/peptide receptor; protein/protein receptor; peptide nucleic acid/complementary strand; enzyme substrate.enzyme; DNA or RNA/cDNA or cRNA; hormone/hormone receptor; and ion/chelator.

Alternatively, a monomer, labeled with a dark quenching compound can be incorporated into a polymer labeled with a luminophore, resulting in quenching of luminescence. In particular, a dark quenching compound-labeled nucleotide can be incorporated via the polymerase chain reaction into a double stranded DNA molecular that is labeled with a luminophore.

In another embodiment, the initially quenched luminescence of a luminophore associated becomes dequenched upon being released from the constraint of being in close proximity to a dark quenching compound. The quenching compound is optionally associated with the same molecular structure as the luminophore, or the donor and acceptor are associated with adjacent but distinct subunits of the structure. The following systems, among others, can be analyzed using energy transfer pairs to detect and/or quantify structural disassembly: detection of protease activity using fluorogenic substrates (for example HIV protease assays); detection of enzyme-mediated protein modification (e.g., cleavage of carbohydrates/fatty acids, phosphates, prosthetic groups); immunoassays (via displacement/competitive assays); detection of DNA duplex unwinding (e.g. helicase/topoisomerase/gyrase assays); nucleic acid strand displacement; ds DNA melting; nuclease activity; lipid distribution and transport; and TAQMAN assays.

Structural disassembly is typically detected by observing the partial or complete restoration of luminescence, as a conjugated substance is exposed to a degradation conditions of interest for a period of time sufficient for degradation to occur. A restoration of luminescence indicates an increase in separation distance between the luminophore and quenching compound, and therefore a degradation of the conjugated substance. If the detectable difference in luminescence is detected as the degradation proceeds, the assay is a continuous assay. Since most enzymes show some selectivity among substrates, and as that selectivity can be demonstrated by determining the kinetic differences in their hydrolytic rates, rapid testing for the presence and activity of the target enzyme is provided by the enhancement of luminescence of the labeled substrate following separation from the quenching compound.

In another embodiment of the invention, a single-stranded oligonucleotide signal primer is labeled with both a dark quenching compound and a fluorescent donor dye, and incorporates a restriction endonuclease recognition site located between the donor dye and the quenching compound. The single-stranded oligonucleotide is not cleavable by a restriction endonuclease enzyme, but upon binding to a complementary (target) nucleic acid, the resulting double stranded nucleic acid is cleaved by the enzyme and the decreased quenching is used to detect the presence of the complementary nucleic acid (See, for example, U.S. Pat. No. 5,846,726).

A single nucleotide polymorphism (SNP) can also be detected through the use of sequence specific primers, by detection of melt temperatures of the double stranded nucleic acid. In this aspect, the complementary or substantially complementary strands are labeled with a dark quenching compound and a luminophore donor, respectively, and dissociation of the two strands (melting) is detected by the restoration of luminescence of the donor.

In yet another example, the rupture of a vesicle containing a highly concentrated solution of luminophores and quenching compounds is readily detected by the restoration of luminescence after the vesicle contents have been diluted sufficiently to minimize quenching.

The dark quenching compound and the fluorescent donor can be present on the same or different substances, and a change in the three-dimensional structural conformation of one or more components of the assay can result in either luminescence quenching or restoration of luminescence, typically by substantially decreasing or increasing the separation distance between the quenching compound and a luminophore. The following systems, among others, can be analyzed using energy transfer pairs to detect and/or quantify conformation changes: protein conformational changes; protein folding; structure and conformation of nucleic acids; drug delivery; antisense oligonucleotides; and cell-cell fusion (e.g. via the diffusion apart of an initial donor-quenching compound pair). By conformation change is meant, for example, a change in conformation for an oligonucleotide upon binding to a complementary nucleic acid strand. In one such assay, labeled oligonucleotides are substantially quenched when in solution, but upon binding to a complementary strand of nucleic acid become highly fluorescent(See, for example, European Patent Application EP 0 745 690). The change in conformation can occur when an oligonucleotide that has been labeled at its ends with a quenching compound and a luminophore, respectively, loses its G-quartet conformation upon hybridization to a complementary sequence resulting in decreased luminescence quenching (See, for example, U.S. Pat. No. 5,691,145). Alternatively, the binding of an enzyme substrate within the active site of a labeled enzyme may result in a change in tertiary or quaternary structure of the enzyme, with restoration or quenching of luminescence.

Kits that facilitate the practice of the methods of the invention as described above are also provided. The kits of the invention can comprise a dark quenching compound. The dark quenching compound is preferably present conjugated to a biological molecule (e.g., a nucleotide, oligonucleotide, nucleic acid polymer, peptide, or protein). The kit can further comprise one or more buffering agents, typically present as an aqueous solution.

According to one embodiment, the kit comprises a dark quenching compound and a luminescent donor. The quenching compound and luminescent donor can each be a part of a conjugate or can be present in solution as free compounds. Such a kit can be used for the detection of cell-cell fusion, as fusion of a cell containing the quenching compound with a cell containing a luminescent donor would result in quenching of luminescence. Conjugation of either the quenching compound or the luminescent donor or both to biomolecules, such as polysaccharides, would help retain the reagents in their respective cells until cell fusion occurred.

In another embodiment, the kit comprises a dark quenching compound and a luminescent donor, each conjugated to a complementary member of a specific binding pair. In this aspect of the invention, binding of the two specific binding pair members results in quenching of luminescence. The kit can be used for the detection of competitive binding to one or the other specific binding pair members, or for the detection of an environmental condition or component that either facilitates or inhibits binding of the specific binding pair members.

In another embodiment, the kit comprises a conjugate of a quenching compound and a conjugate of a luminescent donor, wherein the conjugates are selected such that the action of a particular enzyme results in covalent or noncovalent association of the two conjugates, resulting in quenching of fluorescence. Where the conjugated substances are nucleotides, oligonucleotides or nucleic acid polymers, the kit can be used for the detection of, for example, ligase, telomerase, helicase, topoisomerase, gyrase, DNA/RNA polymerase, or reverse transcriptase enzymes.

In another embodiment, the kit comprises a biomolecule that is covalently labeled by both a dark quenching compound and a luminescent donor. The labeled biomolecule can exhibit luminescence until a specified environmental condition (such as the presence of a complementary specific binding pair) causes a conformation change in the biomolecule resulting in the quenching of luminescence. Alternatively, the biomolecule can be initially quenched and a specified environmental condition, such as the presence of an appropriate enzyme or chemical compound, can result in degradation of the biomolecule and restoration of luminescence. Such a kit would can be used for the detection of complementary oligonucleotide sequences or for the detection of enzymes such as nuclease, lipase, protease, or cellulase.

While the foregoing specification teaches the principles of the present invention, with examples provided for the purpose of illustration, it will be appreciated by one skilled in the art from reading this disclosure that various changes in form and detail can be made without departing from the true scope of the invention.

1. A compound having a general structure as set forth in formulae (Ia), (1b) or (II): 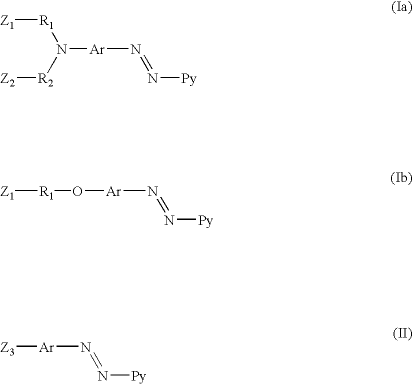
wherein:
Ar is a substituted or non-substituted aryl group;
Py is a substituted or non-substituted hetero-aromatic ring;
R₁ and R₂ independently represent a C₁ to C₄ alkyl chain or hydrogen;
Z₁ and Z₂ independently represent a substituted or non-substituted sulfonate, phosphate or carboxylate, pentafluorophenyl ester, p-nitrophenylester, or a moiety represented by one of the following formulae: 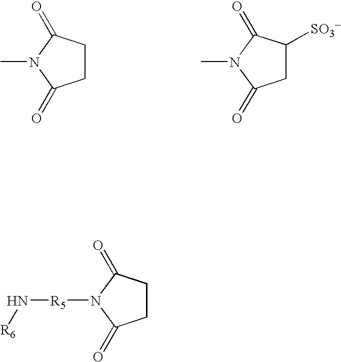
wherein R₅ and R₆ are alkyl groups;
Z₃ is OH, OR₇, NH₂, NHAr′ or NAr′₂, SH, SR₇, or SCN wherein Z₃ is at the ortho-position of the aryl group Ar, Ar′ is an aromatic or hetroaromatic ring and R₇ is an alkyl or aromatic group.

2. The compound of claim 1 having a general structure as set forth in formulae (IIIa), (IIIb) or (IV) below: 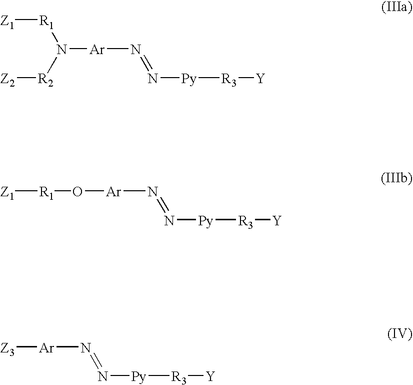
wherein:
R₃ is a C₁ to C₈ alkyl chain; and
Y is: —COOH, —SH, —OH, isocyanate, epoxide, iodoacetate, bromoacetate, NR′R″ where R′ and R″ are hydrogen or alkyl or aromatic rings, or —COOR₄ wherein R₄ is pentafluorophenyl ester, p-nitrophenylester, or a moiety represented by one of the following formulae: 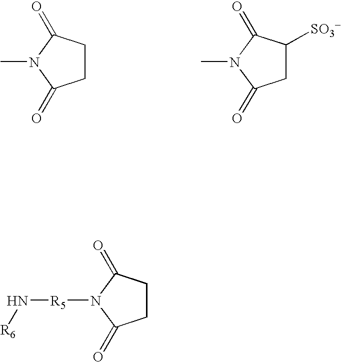
wherein R₅ and R₆ are alkyl groups or wherein Y is a moiety represented by the following formula:
—OP(OR₈)(N(R₉)₂)₂
wherein, R₈ and R₉ are alkyl and substituted alkyl.

3. The compound of claim 2, wherein Y is a moiety represented by the formula —OP(OR₈)(N(R₉)₂)₂ wherein R₈ is cyanoethyl and R₉ is isopropyl.

4. The compound of claim 1 having a structure represented by either of the following formulae:

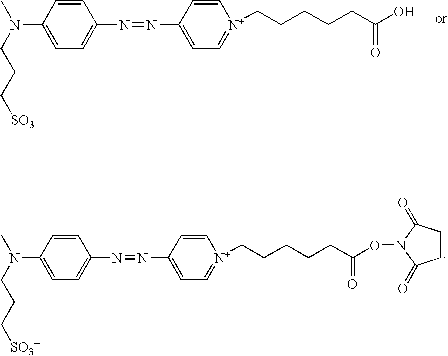

5. A bioconjugate comprising a biomolecule conjugated to a quencher compound having a structure as set forth in claim 1.

6. The bioconjugate of claim 5, wherein the biomolecule is a polypeptide, a protein, an antibody, or a nucleic acid.

7. The bioconjugate of claim 5, wherein the biomolecule is a nucleic acid.

8. The bioconjugate of claim 5, further comprising a fluorescer conjugated to the biomolecule, wherein the quencher compound quenches the fluorescence from the fluorescer when associated therewith.

9. A metal complex comprising a metal containing compound complexed to a quencher compound having a structure as set forth in claim 1.

10. An assay for determining the presence and/or amount of an analyte in a sample comprising:
combining a bioconjugate as set forth in claim 5 and a fluorescer with the sample, wherein the quencher compound of the bioconjugate quenches the fluorescence of the fluorescer when associated therewith; and
detecting a change in fluorescence.

11. The assay of claim 10, wherein the analyte is labeled with the fluorescer.

12. The assay of claim 11, wherein the analyte associates with the biomolecule of the bioconjugate resulting in a decrease in fluorescence.

13. The assay of claim 11, wherein the analyte associates with a biomolecule in a sample and wherein association of the analyte and the biomolecule results in an increase in fluorescence.

14. The assay of claim 13, wherein the fluorescer is conjugated to the bioconjugate.

15. The assay of claim 14, wherein:
the analyte is a single stranded nucleic acid;
the biomolecule of the bioconjugate comprises a single stranded nucleic acid which hybridizes to the analyte; and
wherein hybridization of the analyte and the biomolecule of the bioconjugate results in separation of the quencher compound and the fluorescer resulting in an increase in fluorescence.

16. The assay of claim 14, wherein:
the analyte is an enzyme;
the biomolecule of the bioconjugate comprises a polypeptide substrate for the enzyme; and
wherein association of the analyte and the bioconjugate comprises enzymatic degradation of the polypeptide substrate resulting in separation of the fluorescer from the quencher and an increase in fluorecsnce.

17. The assay of claim 14, wherein:
the analyte is a single stranded nucleic acid;
the biomolecule of the bioconjugate comprises a single stranded nucleic acid which hybridizes to the analyte and which includes a restriction endonuclease recognition site;
the method further comprising combining a restriction endonuclease enzyme with the sample, wherein the enzyme can cleave the nucleic acid at the recognition site only when the nucleic acid is hybridized to the analyte.

18. The assay of claim 10, wherein the fluorescer is conjugated to a second biomolecule.

19. The assay of claim 18, wherein the analyte, the biomolecule of the bioconjugate and the second biomolecule each comprise single stranded nucleic acids and wherein the biomolecule of the bioconjugate hybridizes to the second biomolecule and wherein the analyte hybridizes to either of the biomolecule of the bioconjugate or the second biomolecule.

20. A method of detecting a single nucleotide polymorphism (SNP) of a target nucleic acid comprising:
combining a first bioconjugate with a sample comprising nucleic acids, the first bioconjugate comprising a first single stranded nucleic acid primer for a target nucleic acid, the first single stranded nucleic acid primer labeled with a quenching compound having a structure as set forth in claim 1;
combining a second bioconjugate with the sample, the second bioconjugate comprising a second single stranded nucleic acid primer for the target nucleic acid, the second single stranded nucleic acid primer labeled with a fluorescer, wherein the fluorescer is quenched by the quenching compound when the first and second primers are hybridized to the target nucleic acid;
allowing the first and second primers to hybridize to nucleic acids in the sample;
increasing the temperature of the sample; and
observing a change in fluorescence of the sample;
wherein an increase in fluorescence upon heating indicates melting of the hybridized strands and wherein the temperature at which fluorescence is observed is an indication of the presence and/or amount of the SNP in the sample.

21. An assay as set forth in claim 10, wherein the change in fluorescence results from a change in conformation of the biomolecule of the bioconjugate or of an assembly comprising the biomolecule of the bioconjugate.
